(19) United States
(12) Patent Application Publication
(10) Pub. No.: US 2014/0221840 A1
(43) Pub. Date: Aug. 7, 2014

(54) ULTRASOUND TRANSDUCER, ULTRASOUND PROBE INCLUDING THE SAME, AND ULTRASOUND DIAGNOSTIC EQUIPMENT INCLUDING THE ULTRASOUND PROBE

(71) Applicant: SAMSUNG MEDISON CO., LTD., Gangwon-do (KR)

(72) Inventors: Jong-sun KO, Gangwon-do (KR); Gil-ju JIN, Gangwon-do (KR); Jung-lim PARK, Gangwon-do (KR); Dong-hyun KIM, Gangwon-do (KR)

(73) Assignee: SAMSUNG MEDISON CO., LTD., Gangwon-do (KR)

(21) Appl. No.: 14/037,014

(22) Filed: Sep. 25, 2013

(30) Foreign Application Priority Data
Feb. 5, 2013 (KR) 10-2013-0012943

(51) Int. Cl. A61B 8/00; B06B 1/06

(52) U.S. Cl. CPC A61B 8/4483; A61B 8/4444; B06B 1/0622
USPC 600/459; 310/336

(57) ABSTRACT

Provided are an ultrasound transducer, an ultrasound probe including the ultrasound transducer, and ultrasound diagnostic equipment including the ultrasound probe. The ultrasound transducer includes a piezoelectric unit including a plurality of piezoelectric elements which vibrate to convert ultrasound waves into electrical signals and electrical signals back into ultrasound waves, and a dummy piezoelectric unit which is disposed at edges of the effective piezoelectric unit and includes a plurality of dummy piezoelectric elements which vibrate due to vibration of the piezoelectric unit.

### CROSS-REFERENCE TO RELATED PATENT APPLICATION

This application claims the benefit of Korean Patent Application No. 10-2013-0012943, filed on Feb. 5, 2013, in the Korean Intellectual Property Office, the disclosure of which is incorporated herein in its entirety by reference.

### BACKGROUND OF THE INVENTION

1. Field of the Invention

The present invention relates to an ultrasound transducer including dummy piezoelectric elements, an ultrasound probe including the ultrasound transducer, and ultrasound diagnostic equipment including the ultrasound probe.

2. Description of the Related Art

In general, ultrasound diagnostic equipment is used to diagnose a disease by sending ultrasound waves to an organ within a human body or an animal body, detecting an echo signal reflected from the organ, displaying a cross-sectional image of the organ on a monitor, and providing information necessary for diagnosing the disease.

For this purpose, the ultrasound diagnostic equipment includes an ultrasound probe for transmitting ultrasound waves to the organ and receiving an echo signal from the organ.

The ultrasound probe includes an ultrasound transducer that converts an ultrasound signal into an electrical signal and vice versa. A typical ultrasound transducer includes a plurality of piezoelectric elements.

Thus, the ultrasound diagnostic equipment configured as described above radiates an ultrasound wave into the object and converts the reflected ultrasound signal into an electrical signal, and generates an ultrasound image in response to the electrical signal.

According to this process, such ultrasound diagnostic equipment including the ultrasound probe is used in a wide variety of medical applications, such as detection of foreign materials within a human body, measurement of a degree of a lesion, observation of tumors and fetuses, etc.

In this regard, since the piezoelectric elements are arranged adjacent to one another, crosstalk is caused between neighboring piezoelectric elements due to vibrations thereof. In particular, since an acoustic crosstalk between central piezoelectric elements is different from crosstalk between lateral piezoelectric elements, it may be difficult to create an accurate ultrasound image of the investigated organ. Accordingly, there is a need for an ultrasound transducer, an ultrasound probe, and ultrasound medical equipment that can produce accurate ultrasound images to facilitate medical diagnosis and other various purposes.

### SUMMARY OF THE INVENTION

The present invention provides an ultrasound transducer having a dummy piezoelectric element, an ultrasound probe including the ultrasound transducer, and ultrasound diagnostic equipment including the ultrasound probe.

According to an aspect of the present invention, there is provided an ultrasound transducer including a piezoelectric unit including a plurality of piezoelectric elements which vibrate to convert ultrasound waves into electrical signals and electrical signals back into ultrasound waves, and a dummy piezoelectric unit which is disposed at edges of the effective piezoelectric unit and includes a plurality of dummy piezoelectric elements which vibrate due to vibration of the piezoelectric unit.

The piezoelectric elements may have the same shape as the dummy piezoelectric elements.

A kerf between adjacent dummy piezoelectric elements or the dummy piezoelectric elements and the corresponding piezoelectric elements may be greater than 0.12 times the wavelength of ultrasound waves.

A pitch between adjacent dummy piezoelectric elements or the dummy piezoelectric elements and the corresponding piezoelectric elements may be greater than 1.5 times the wavelength of ultrasound waves.

The dummy piezoelectric unit may have a width s greater than twice the wavelength of the ultrasound waves.

The plurality of piezoelectric elements may be arranged in a one-dimensional (1 D) array, and the plurality of dummy elements may be arranged in a 1D array with the plurality of piezoelectric elements interposed therebetween.

The plurality of piezoelectric elements may be arranged in a two-dimensional (2D) array, and the plurality of dummy elements may be arranged in a 2D array to surround the plurality of piezoelectric elements.

The transducer may further include a backing member which supports the piezoelectric units and the dummy piezoelectric units and absorbs the ultrasound waves.

The backing member may have a plurality of trenches formed in a region that is not in contact with the plurality of piezoelectric elements and the plurality of dummy piezoelectric elements.

A depth of the trenches formed in a region corresponding to the dummy piezoelectric unit may be greater than 0.1 times the wavelength of ultrasound waves.

The depth of the plurality of trenches may increase away from the piezoelectric unit.

The transducer may further include a matching unit for matching an acoustic impedance of the ultrasound waves produced by the piezoelectric unit with an acoustic impedance of an object under examination.

The matching unit may include a plurality of separate matching elements respectively disposed on at least one of the piezoelectric unit and the dummy piezoelectric unit corresponding thereto.

Crosstalk between the plurality of piezoelectric elements may be uniform.

According to another aspect of the present invention, there is provided an ultrasound probe including a housing and the ultrasound transducer described above, wherein the ultrasound transducer is accommodated in the housing.

According to another aspect of the present invention, there is provided ultrasound diagnostic equipment including the above-described ultrasound probe and a signal processor which creates an ultrasound image in response to an electrical signal corresponding to an ultrasound echo signal received by the ultrasound probe.

The ultrasound diagnostic equipment may further include an ultrasound controller for controlling the ultrasound probe so as to generate ultrasound waves. The ultrasound controller may apply an electrical signal only to the piezoelectric unit without applying the electrical signal to the dummy piezoelectric unit.

The signal processor may determine an electrical signal received from one of the piezoelectric elements in the effective piezoelectric unit as being an electrical signal corresponding to an ultrasound echo signal received by the piezoelectric element.

The signal processor may also determine an average of electrical signals received by a first piezoelectric element in the piezoelectric unit and at least one of a piezoelectric element and a dummy piezoelectric element adjacent to the first piezoelectric element as being an electrical signal corresponding to an ultrasound echo signal received by the first piezoelectric element.

### BRIEF DESCRIPTION OF THE DRAWINGS

The above and other features and advantages of the present invention will become more apparent by describing in detail exemplary embodiments thereof with reference to the attached drawings in which:

FIG. 1 is a front view of ultrasound diagnostic equipment including an ultrasound probe according to an embodiment of the present invention;

FIG. 2 is a perspective view schematically illustrating an internal structure of an ultrasound probe according to an embodiment of the present invention;

FIGS. 3A and 3B illustrate arrangements of piezoelectric elements according to an embodiment of the present invention;

FIG. 4 illustrates an ultrasound transducer according to another embodiment of the present invention;

FIG. 5 is a block diagram of ultrasound diagnostic equipment according to an embodiment of the present invention;

FIGS. 6A and 6B illustrate experimental results showing changes in an acoustic signal according to the presence of a dummy piezoelectric unit;

FIG. 7 illustrates experimental results showing changes in an acoustic signal with respect to a width of a dummy piezoelectric unit;

FIG. 8 illustrates experimental results showing changes in an acoustic signal with respect to a kerf width of a dummy piezoelectric unit; and

FIG. 9 illustrates experimental results showing changes in an acoustic signal with respect to a depth of a trench of a dummy piezoelectric unit.

### DETAILED DESCRIPTION OF THE INVENTION

A backing member, a transducer including the backing member, and an ultrasound probe including the transducer according to example embodiments will be described in detail with reference to the accompanying drawings. In the drawings, like reference numerals denote like elements. Expressions such as “at least one of,” when preceding a list of elements, modify the entire list of elements and do not modify the individual elements of the list.

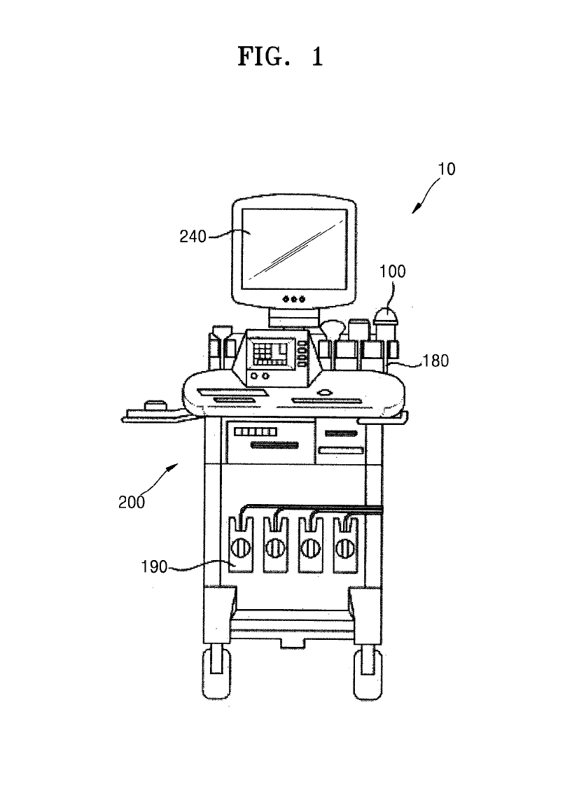

FIG. 1 is a front view of ultrasound diagnostic equipment 10 including an ultrasound probe 100 according to an embodiment of the present invention. The ultrasound probe 10 may be used in an ultrasound diagnostic system and other various ultrasound devices. Hereinafter, for convenience of explanation, it is assumed that the ultrasound probe 100 is used in ultrasound diagnostic equipment.

Referring to FIG. 1, the ultrasound diagnostic equipment 10 includes a main body 200 including manipulation buttons and a display 240 whereon an image of an object under examination is displayed and the ultrasound probe 100 which emits ultrasound waves toward the object and receives an ultrasound echo signal from the object. The ultrasound probe 100 is connected to the main body 200 by a cable 180 and a connector 190.

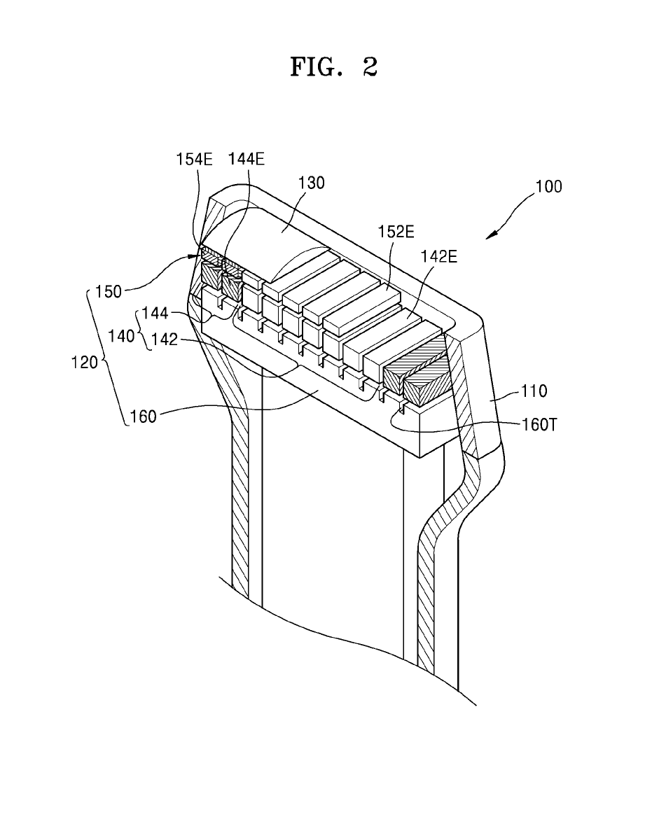

FIG. 2 is a partially broken away perspective view schematically illustrating an internal structure of the ultrasound probe 100 according to an embodiment of the present invention;

Referring to FIG. 2, the ultrasound probe 100 includes a housing 110 and an ultrasound transducer 120 that is accommodated in the housing and generates ultrasound waves when a voltage is applied thereto from the ultrasound diagnostic equipment 10. The ultrasound probe 100 may further include an acoustic lens 130 for focusing the ultrasound waves.

The ultrasound transducer 120 includes a piezoelectric unit 140 which converts an electrical signal into ultrasound waves and vice versa, a matching unit 150 which matches an acoustic impedance of the ultrasound waves produced by the piezoelectric unit 140 with an acoustic impedance of the object, and a backing member 160 which absorbs ultrasound waves transmitted in an opposite direction to the object.

When the piezoelectric unit 140 vibrates, an electrical signal is converted into ultrasound waves or ultrasound waves are converted back into an electrical signal. The piezoelectric unit 140 includes a plurality of piezoelectric elements 142E and 144E. The piezoelectric elements 142E and 144E may be formed by dividing a piezoelectric material into a plurality of elements. For example, a piezoelectric material elongated in a width direction may be subjected to a dicing process. However, the present invention is not limited thereto, and the plurality of piezoelectric elements 142E and 144E may be formed by using other various methods, e.g., by pressing a piezoelectric material into a metal mold. The piezoelectric material may be a material exhibiting a piezoelectric effect, such as a piezoelectric ceramic, a single crystal, or a piezoelectric composite material made by combining a piezoelectric ceramic or a single crystal with a polymer.

The piezoelectric unit 140 is divided into a piezoelectric region 142 and a dummy piezoelectric region 144. The piezoelectric region 142 includes piezoelectric elements 142E which vibrate so as to convert ultrasound waves into an electrical signal and vice versa. The dummy piezoelectric region 144 includes dummy piezoelectric elements 144E that vibrate due to vibrations of the piezoelectric elements 142E. The dummy piezoelectric region 144 is disposed at edges of the effective piezoelectric region 142. The piezoelectric elements 142E and the dummy piezoelectric elements 144E are described in more detail below.

The matching unit 150 is disposed on a front surface of the piezoelectric unit 140 and gradually changes an acoustic impedance of the ultrasound waves generated by the piezoelectric unit 140 to approximately match an acoustic impedance of the object. In this case, the front surface of the piezoelectric unit 140 may be one of the surfaces of the piezoelectric unit 140 which is closest to the object, and a rear surface thereof may be a surface opposite the front surface.

The matching unit 150 may include a plurality of matching elements 152E and 154E disposed on the front surfaces of the piezoelectric elements 142E and the dummy piezoelectric elements 144E corresponding thereto. However, the present invention is not limited thereto, and the matching unit 150 may be elongated along the front surface of the piezoelectric unit 140. Although the matching unit 150 has a single layer structure in the present embodiment, the matching unit 150 may have a multilayer structure.

The backing member 160 is disposed on a rear surface of the piezoelectric unit 140 so as to support the piezoelectric elements 142E and the dummy piezoelectric elements 144E and may absorb ultrasound waves which are transmitted to a back side of the piezoelectric unit 140 and not directly used for examination or diagnosis. The backing member 160 may have the same width in a width direction of the piezoelectric unit 140. In this case, the width direction is a direction along a longer edge of the piezoelectric unit 140. The backing member 160 has a plurality of electrodes (not shown) disposed therein for applying a voltage to the piezoelectric unit 140. Since each of the plurality of electrodes is coupled to a corresponding one of the piezoelectric elements 142E and the dummy piezoelectric elements 144E, the number of the plurality of electrodes may be equal to the number of the piezoelectric elements 142E and the dummy piezoelectric elements 144E. The plurality of electrodes may be connected only to the piezoelectric elements 144E or both of the piezoelectric elements 142E and the dummy piezoelectric elements 144E.

The backing member 160 has a plurality of trenches 160T formed in a region which is not in contact with the piezoelectric elements 142E and the dummy piezoelectric elements 144E. The plurality of trenches 160T are formed along an inward direction of the backing member 160, which is opposite the direction of the piezoelectric unit 140. The trenches 160T serve to reduce the effect of vibrations of each of the piezoelectric elements 142E and the dummy piezoelectric elements 144E on adjacent elements.

The acoustic lens 130 is disposed on a front surface of the ultrasound transducer 120 and condenses ultrasonic waves generated by the piezoelectric unit 140. The acoustic lens 130 may be formed of a silicon rubber having an acoustic impedance close to that of the object. The acoustic lens 130 may have a central portion of a convex or flat shape or other various shapes according to design requirements.

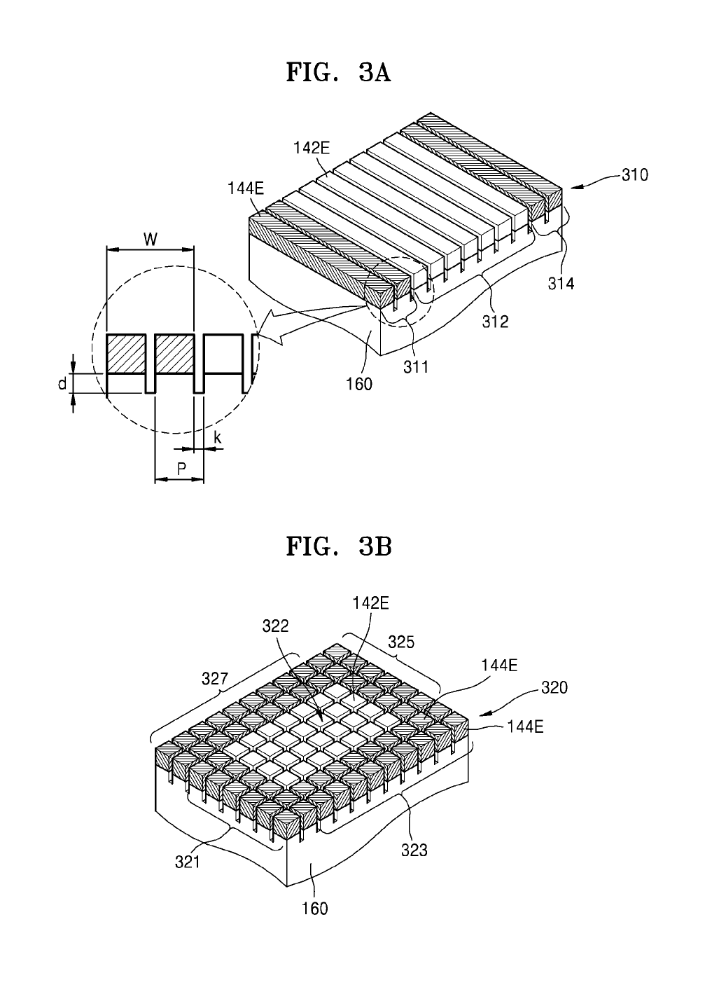

FIGS. 3A and 3B illustrate arrangements of the piezoelectric elements 142E and the dummy piezoelectric elements 144E according to an embodiment of the present invention.

Referring to FIG. 3A, the plurality of piezoelectric elements 142E and dummy piezoelectric elements 144E are arranged on a front surface of the backing member 160 in a one-dimensional (1D) array in a width direction of a piezoelectric unit 310. This is referred to as a 1D piezoelectric unit 310. The 1D piezoelectric unit 310 may be a linear or curved array. The shape of the array may vary depending on design requirements. Although the cost of manufacturing the 1D piezoelectric unit 310 may be low, creating a 3D stereoscopic image by using the 1D piezoelectric unit 310 may be difficult.

More specifically, the plurality of piezoelectric elements 142E are arranged in a 1D array. The plurality of dummy piezoelectric elements 144E are divided into first and second dummy piezoelectric units 311 and 314 and arranged in a 1D array with the plurality of piezoelectric elements 142 being sandwiched between the first and second dummy piezoelectric units 311 and 314. The first dummy piezoelectric unit 311 including one or more dummy piezoelectric elements 144E, a piezoelectric unit 312 including one ore more piezoelectric elements 142E, and the second dummy piezoelectric unit 314 including one or more dummy piezoelectric elements 144E may be sequentially arranged in a 1D array in the width direction of the 1D piezoelectric unit 310. The first and second dummy piezoelectric units 311 and 314 may be disposed with the piezoelectric unit 312 interposed therebetween, thereby making acoustic crosstalk between the effective piezoelectric elements 142E uniform.

A kerf K between adjacent piezoelectric elements 142E and dummy piezoelectric elements 144E may be greater than 0.12 times the wavelength of an ultrasound wave generated by the 1D piezoelectric unit 310. In particular, when the kerf K between adjacent piezoelectric elements 142E and dummy piezoelectric elements 144E is greater than 0.12 times the wavelength of an ultrasound wave, a difference in acoustic crosstalk between the piezoelectric elements 142E at edges of the piezoelectric unit 142 and at a central portion thereof may be reduced.

A pitch P between the piezoelectric elements 142E and dummy piezoelectric elements 144E may be greater than 0.5 times the wavelength of an ultrasound wave generated by the 1D piezoelectric unit 310. In particular, when a pitch P between the piezoelectric elements 142E and dummy piezoelectric elements 144E is greater than 0.5 times the wavelength of an ultrasound wave, a difference in acoustic crosstalk between the piezoelectric elements 142E at edges of the piezoelectric unit 312 and at the central portion thereof may be reduced.

Furthermore, a width W of the dummy piezoelectric unit 311 or 314 may be 1.5 times the wavelength of an ultrasound wave generated by the piezoelectric unit 312. In particular, when a width W of the dummy piezoelectric unit 311 or 314 is 1.5 times the wavelength of an ultrasound wave, a difference in acoustic crosstalk between the piezoelectric elements 142E at edges of the piezoelectric unit 312 and at the central portion thereof may be reduced.

Referring to FIG. 3B, a plurality of piezoelectric elements 142E and dummy piezoelectric elements 144E may be arranged in a two-dimensional (2D) array in a width direction as well as in a direction perpendicular to the width direction, which is referred to as a 2D piezoelectric unit 320. The 2D piezoelectric unit 320 may be a linear or curved array. The shape of the array may vary depending on design requirements. In this case, the 2D piezoelectric unit 320 transmits ultrasonic waves to the object by appropriately delaying an input time for signals input to the respective piezoelectric elements 142E and dummy piezoelectric elements 144E and receives a plurality of echo signals from the object. The plurality of echo signals are used to form a 3D image.

If the number of the piezoelectric elements 142E and dummy piezoelectric elements 144E is increased, ultrasonic images of higher quality may be obtained. However, in order to increase the number of the piezoelectric elements 142E and the dummy piezoelectric elements 144E, the size thereof has to be reduced, and thus, a large amount of crosstalk between piezoelectric elements disposed in a narrow region and a large difference in acoustic crosstalk between the piezoelectric elements 142E and the dummy piezoelectric elements 144E disposed at edges and a central portion of the 2D piezoelectric unit 320 may be generated.

The plurality of piezoelectric elements 142E are arranged in a 2D array while the plurality of dummy piezoelectric elements 144E are arranged in a 2D array so as to surround the plurality of piezoelectric elements 142E. For example, a piezoelectric unit 322 including a 2D array of the piezoelectric elements 142E may be disposed at a central portion of the 2D piezoelectric unit 320. First through fourth dummy piezoelectric units 321, 323, 325, and 327 may be disposed along edges of the 2D piezoelectric unit so as to surround the piezoelectric unit 322.

A kerf K between the piezoelectric elements 142E and the dummy piezoelectric elements 144E may be greater than 0.12 times the wavelength of an ultrasound wave generated by the 2D piezoelectric unit 320. A pitch P between the piezoelectric elements 142E and dummy piezoelectric elements 144E may be greater than 0.5 times the wavelength of an ultrasound wave generated by the 2D piezoelectric unit 320. A width W of the dummy piezoelectric unit 321, 323, 325, or 327 may be greater than twice the wavelength of an ultrasound wave generated by the 2D piezoelectric unit 320.

The trenches 160T formed in the backing member 160 have a uniform depth d. However, the present invention is not limited thereto and the depth d of the trenches 160T may vary depending on whether the backing member 160 supports dummy piezoelectric units. For example, the trenches 160T in a dummy backing member have a depth greater than that of the trenches 160T in a backing member. Alternatively, the trenches 160T in the dummy backing member may have different depths according to their distances from the backing member.

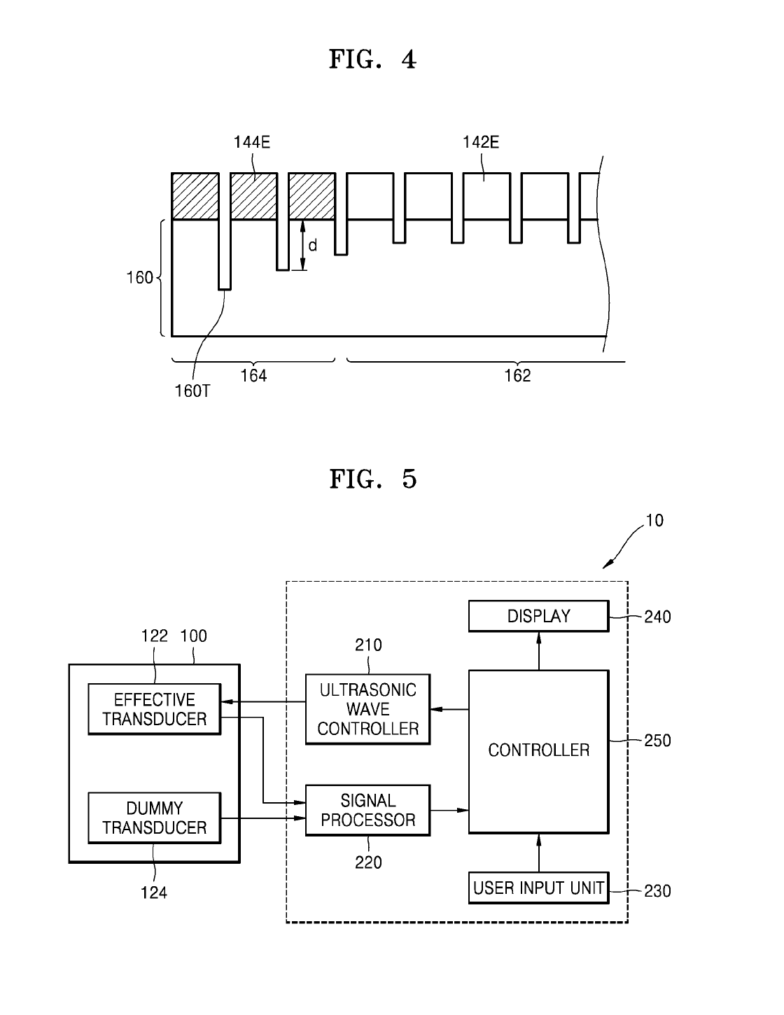

FIG. 4 illustrates an ultrasound transducer according to another embodiment of the present invention. Referring to FIG. 4, trenches 160T formed in a backing member 162 have a uniform depth d. On the other hand, trenches 160T formed in a dummy backing member 164 have a depth which increases away from the backing member 162. Thus, the effect of vibrations of a dummy piezoelectric unit 144 on a piezoelectric unit 142 may be minimized.

FIG. 5 is a block diagram of ultrasound diagnostic equipment 10 of FIG. 1 according to an embodiment of the present invention. Referring to FIG. 5, the ultrasound diagnostic equipment 10 includes an ultrasound probe 100 which sends ultrasound waves to a object under examination and receives echoes of the ultrasonic waves from the object and a main body 200 that uses signals received from the ultrasound probe 100 to create an ultrasound image of the object. The main body 200 may be connected to the ultrasound probe 100 in a wired or wireless manner.

The ultrasound probe 100 includes a transducer 122 which vibrates so as to convert ultrasound waves into electrical signals and electrical signals back into ultrasound waves and a dummy transducer 124 which vibrates due to the vibration of the transducer 122.

The transducer 122 includes a piezoelectric unit (corresponding to the piezoelectric unit 142 in FIG. 2), a portion of a matching unit (corresponding to the matching unit 150 in FIG. 2) disposed above the effective piezoelectric unit, and a portion of a backing member (corresponding to the backing member 160 in FIG. 2) disposed below the piezoelectric unit 142. The dummy transducer 124 includes a dummy piezoelectric unit (corresponding to the dummy piezoelectric unit 144 in FIG. 2), a portion of the matching unit disposed above the dummy piezoelectric unit, and a portion of the backing member disposed below the dummy piezoelectric unit.

The main body 200 includes an ultrasound controller 210 for controlling generation of ultrasound waves, a signal processor 220 which uses ultrasound echo signals to create an ultrasound image, a user input unit 230 which receives a user command for creating an ultrasound image, a display 240 for displaying an ultrasound image or a user command, and a controller 250 for controlling overall operations of the ultrasound diagnostic equipment 10 according to user commands.

The ultrasound controller 210 applies an electrical signal only to the piezoelectric unit in the transducer 122, which then vibrates to convert the electrical signal into an ultrasound wave. The dummy piezoelectric unit may vibrate due to the vibration of the piezoelectric unit.

The signal processor 220 uses an electrical signal corresponding to an ultrasound echo signal to create an ultrasound image. More specifically, the ultrasound probe 100 receives an ultrasound echo signal and converts it to an electrical signal. The signal processor 220 uses only the electrical signal received from the effective transducer 122 to create an ultrasound image. For example, the signal processor 220 may determine the signal received from a piezoelectric element in the transducer 122 as being the electrical signal corresponding to the ultrasound echo signal received by the piezoelectric element. Alternatively, the signal processor 220 uses electrical signals received from the transducer 122 and dummy transducer 124 to create an ultrasound image. For example, an average of electrical signals received by a first piezoelectric element in the transducer 122 and the adjacent piezoelectric element or dummy piezoelectric element may be determined as being an electrical signal corresponding to an ultrasound echo signal received by the first piezoelectric element.

The ultrasound image may be at least one of a brightness (B) mode image representing the intensity of an ultrasound signal according to its brightness, a doppler mode image representing an image of a moving object via a Doppler spectra using the Doppler effect, a motion (M) mode image showing a movement of an object at a predetermined position over time, an elasticity mode image representing response differences between objects with and without compression, and a color (C) mode image representing velocities of moving objects as colors using the Doppler effect.

Since an ultrasound image is created by using a currently known approach, a detailed description thereof is omitted herein. An ultrasound image according to an embodiment of the present invention may include at least one of 1D, 2D, 3D, and 4D images.

The user input unit 230 allows a user to generate input data for controlling the operation of the ultrasound diagnostic equipment 10. The user input unit 230 may include a key pad, a dome switch, a touch pad (resistive/capacitive), a jog wheel, and a jog switch. In particular, when the touch pad forms a layered structure with the display 240, the layered structure may be referred to as a touch screen.

The display 240 displays and outputs information processed by the ultrasound diagnostic equipment 10, such as ultrasound images. When the display 240 and a touch pad with a layer structure are integrated into a touch screen, the display 240 may be used as both output and input devices. The display 240 may include at least one of a liquid crystal display (LCD), a thin film transistor-LCD (TFT-LCD), an organic emitting diode (OLED), a flexible display, and a 3D display. The ultrasound diagnostic equipment 10 may include two or more displays 240 depending on the type of embodiment.

The touch screen may be configured to detect a touch input position, a touched area, and a touch input pressure. Furthermore, the touch screen may be configured to detect a real-touch and a proximity touch.

The components of the ultrasound diagnostic equipment 10 shown in FIG. 5 are not essential ones and are illustrated only for convenience of description. In other words, the ultrasound diagnostic equipment 10 may be realized by using a larger or smaller number of components.

Furthermore, the ultrasonic wave controller 210, the signal processor 220, the user input unit 230, and the controller 250 in the main body 200 are not necessarily separated from the ultrasound probe 100. At least one component in the main body 200 may be included in the ultrasound probe 100. For example, the ultrasound controller 210 or user input unit 230 may be one component of the ultrasound probe 100.

Experimental results showing changes in an acoustic signal according to the presence of a dummy piezoelectric unit are now described. The experiment described below was conducted by using a program called PZFlex.

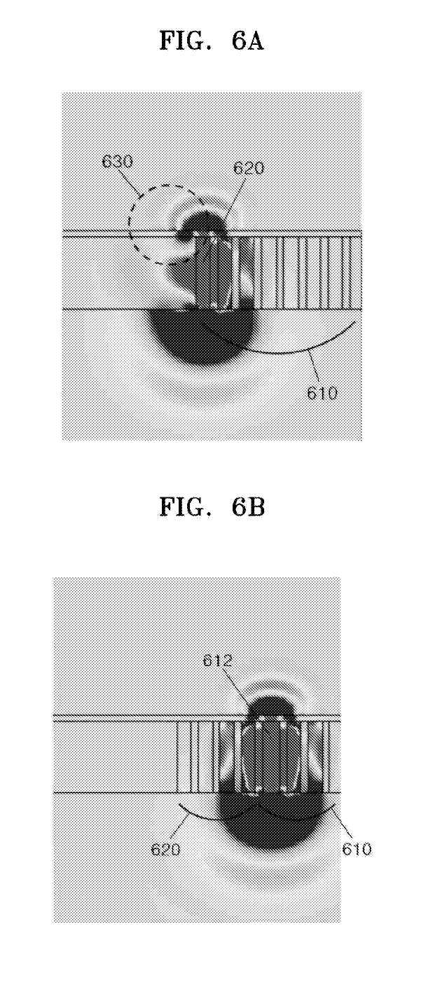

FIGS. 6A and 6B illustrate experimental results showing changes in an acoustic signal according to the presence of a dummy piezoelectric unit.

A piezoelectric unit was designed with a kerf width and a pitch that were 0.16 times the wavelength of an ultrasound wave. An electrical signal was then applied to the piezoelectric unit in order to generate an ultrasound wave having frequency of 1.2 MHz and a velocity of 1,500 m/s. A piezoelectric unit included only the piezoelectric unit, and when an electrical signal was applied to a piezoelectric element disposed at an edge of the piezoelectric unit, the piezoelectric element vibrated to generate ultrasound waves. In this case, as apparent from FIG. 6A, the generated ultrasound waves were not uniform.

On the other hand, a dummy piezoelectric unit was disposed at edges of the piezoelectric unit. The kerf width and pitch between the dummy piezoelectric elements and a depth of a trench were the same as those of the piezoelectric unit. When an electrical signal was applied to a piezoelectric element disposed at an edge of the piezoelectric unit in order to generate an ultrasound wave with frequency of 1.2 MHz and a velocity of 1500 m/s, the ultrasound wave generated at the edge of the piezoelectric unit was uniform as apparent from FIG. 6B. The presence of the dummy piezoelectric unit may make the ultrasound wave uniform.

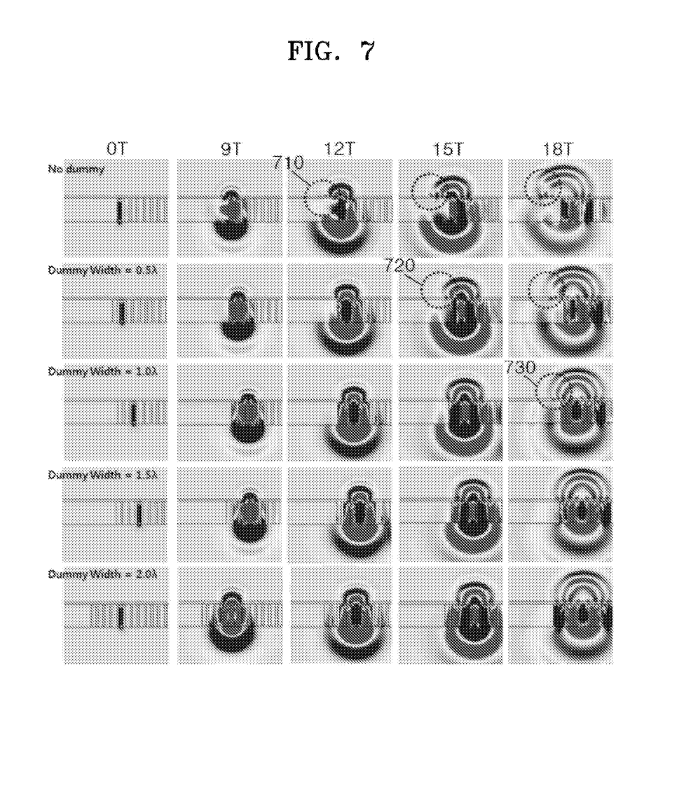

An experiment was conducted to observe changes in an acoustic signal over time by varying a width of a dummy piezoelectric unit while maintaining the same kerf width, pitch, and trench depth of the piezoelectric units and dummy piezoelectric units as described with reference to FIG. 6B. FIG. 7 illustrates experimental results at time intervals of 217 ns after an electrical signal was applied to a piezoelectric element.

Referring to FIG. 7, when a width of the dummy piezoelectric unit was less than 1.5 times the wavelength of an ultrasound wave, the ultrasound wave became uneven after a lapse of a predetermined time following the generation of ultrasound. Conversely, when the width of the dummy piezoelectric unit was greater than 1.5 times the wavelength of the ultrasound wave, the ultrasound wave remained uniform after a lapse of a predetermined time following the generation of ultrasound. Thus, as apparent from FIG. 7, when the width of the dummy piezoelectric unit was greater than 1.5 times the wavelength of the ultrasound wave, acoustic crosstalk was uniform.

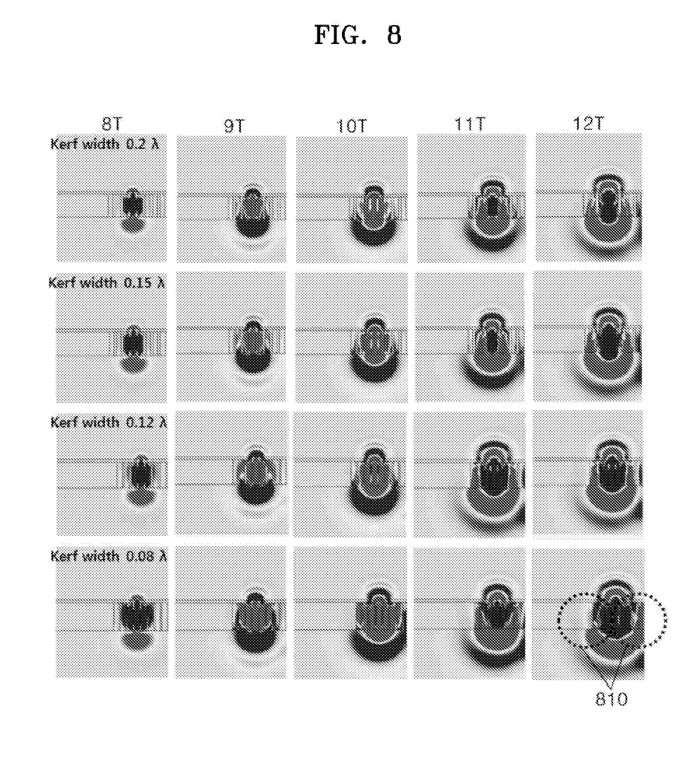

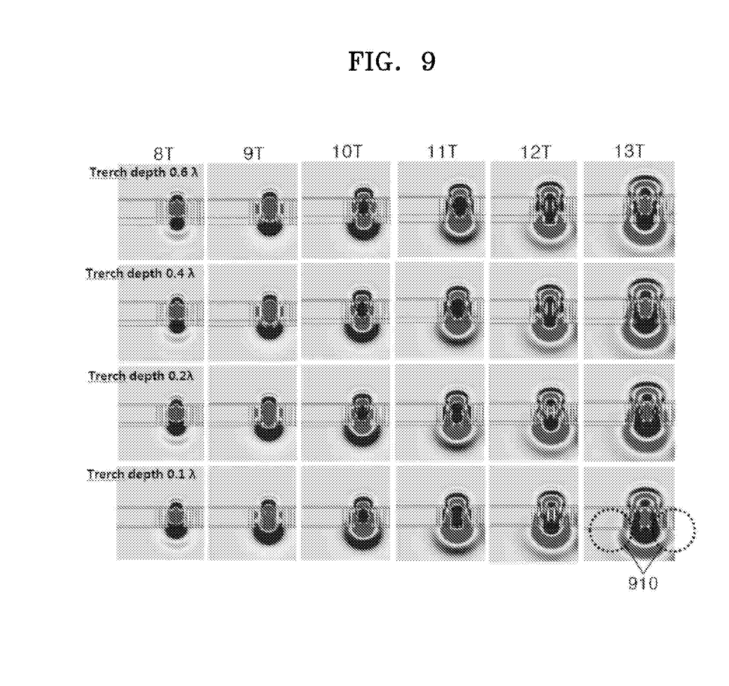

In another experiment, a width of the dummy piezoelectric unit was twice the wavelength of the ultrasound wave while maintaining the same pitch and trench depth as those described with reference to FIG. 6B. The piezoelectric unit had the same kerf width, pitch, and trench depth as those described with reference to FIG. 6B. Changes in an acoustic signal were then observed over time by varying a kerf width of the dummy piezoelectric unit. FIG. 8 illustrates experimental results at time intervals of 217 ns after an electrical signal was applied to a piezoelectric element.

Referring to FIG. 8, when a kerf width of the dummy piezoelectric unit is 0.08 times the wavelength of an ultrasound wave, the ultrasound wave becomes uneven after a lapse of the predetermined time from the generation of the ultrasound. Conversely, when the kerf width of the dummy piezoelectric unit is greater than 0.12 times the wavelength of the ultrasound wave, the ultrasound wave remains uniform after a lapse of the predetermined time from the generation of the ultrasound. Thus, as apparent from FIG. 8, when the kerf width of the dummy piezoelectric unit is greater than 0.12 times the wavelength of the ultrasound wave, acoustic crosstalk may be uniform.

In another experiment, the dummy piezoelectric unit was designed to have a width twice the wavelength of ultrasound wave while the kerf width and pitch were the same as described with reference to FIG. 6B. Also, the piezoelectric unit had the same kerf width, pitch, and trench depth as described with reference to FIG. 6B. Changes in an acoustic signal were then observed over time by varying a trench depth of the dummy piezoelectric unit. FIG. 9 illustrates experimental results obtained at time intervals of 217 ns after an electrical signal was applied to a piezoelectric element.

Referring to FIG. 9, when a trench depth of the dummy piezoelectric unit is greater than 0.1 times the wavelength of an ultrasound wave, the ultrasound wave becomes uneven after a lapse of the predetermined time from the generation of the ultrasound. Conversely, when the trench depth of the dummy piezoelectric unit is greater than 0.1 times the wavelength of ultrasound wave, the ultrasound wave remains uniform after a lapse of the predetermined time from the generation of the ultrasound. Thus, as apparent from FIG. 9, when the trench depth of the dummy piezoelectric unit is greater than 0.1 times the wavelength of ultrasound wave, acoustic crosstalk may be uniform.

As described above, the use of the dummy piezoelectric unit may reduce acoustic distortions.

While the present invention has been particularly shown and described with reference to exemplary embodiments thereof, it will be understood by those of ordinary skill in the art that various changes in form and details may be made therein without departing from the spirit and scope of the present invention as defined by the following claims. Thus, it should be understood that the exemplary embodiments described therein should be not considered for purposes of limitation, and the present invention should be construed as including all changes, equivalents, and substitutions covered by the spirit and scope thereof.

What is claimed is:

1. An ultrasound transducer comprising:
a piezoelectric unit including a plurality of piezoelectric elements which vibrate to convert ultrasound waves into electrical signals and electrical signals back into ultrasound waves; and
a dummy piezoelectric unit which is disposed at edges of the piezoelectric unit and includes a plurality of dummy piezoelectric elements which vibrate due to vibration of the piezoelectric unit.

2. The transducer of claim 1, wherein the piezoelectric elements have the same shape as the dummy piezoelectric elements.

3. The transducer of claim 1, wherein a kerf between adjacent dummy piezoelectric elements is greater than 0.12 times the wavelength of the ultrasound waves.

4. The transducer of claim 1, wherein a pitch between adjacent dummy piezoelectric elements is greater than 1.5 times the wavelength of the ultrasound waves.

5. The transducer of claim 1, wherein the plurality of piezoelectric elements are arranged in a one-dimensional (1D) array, and the plurality of dummy elements are arranged in a 1D array with the plurality of piezoelectric elements interposed therebetween.

6. The transducer of claim 1, wherein the plurality of piezoelectric elements are arranged in a two-dimensional (2D) array, and the plurality of dummy elements are arranged in a 2D array that surrounds the plurality of piezoelectric elements.

7. The transducer of claim 1, further comprising a backing member which supports the piezoelectric units and dummy piezoelectric units and absorbs the ultrasound waves.

8. The transducer of claim 7, wherein the backing member has a plurality of trenches formed in a region that is not in contact with the plurality of piezoelectric elements and the plurality of dummy piezoelectric elements.

9. The transducer of claim 8, wherein a depth of the trenches formed in a region corresponding to the dummy piezoelectric unit is greater than 0.2 times the wavelength of ultrasound waves.

10. The transducer of claim 8, wherein the depth of the plurality of trenches increases away from the piezoelectric unit.

11. The transducer of claim 1, further comprising a matching unit for matching an acoustic impedance of the ultrasound waves produced by the piezoelectric unit with an acoustic impedance of an object under examination.

12. The transducer of claim 11, wherein the matching unit includes a plurality of separate matching elements respectively disposed on at least one of the piezoelectric unit and the dummy piezoelectric unit corresponding thereto.

13. The transducer of claim 1, wherein crosstalk between the plurality of piezoelectric elements is uniform.

14. An ultrasound probe comprising a housing and the ultrasound transducer of one of claims 1 through 13, wherein the ultrasound transducer is accommodated in the housing.

15. Ultrasound diagnostic equipment comprising the ultrasound probe of claim 14 and a signal processor which creates an ultrasound image in response to an electrical signal corresponding to an ultrasound echo signal received by the ultrasound probe.

16. The ultrasound diagnostic equipment of claim 15, further comprising an ultrasound controller for controlling the ultrasound probe so as to generate ultrasound waves,
wherein the ultrasound controller applies an electrical signal only to the piezoelectric unit, without applying the electrical signal to the dummy piezoelectric unit.

17. The ultrasound diagnostic equipment of claim 15, wherein the signal processor determines an electrical signal received from a piezoelectric element in the piezoelectric unit as being an electrical signal corresponding to an ultrasound echo signal received by the piezoelectric element.

18. The ultrasound diagnostic equipment of claim 15, wherein the signal processor determines an average of electrical signals received by a first piezoelectric element in the piezoelectric unit and at least one of a piezoelectric element and a dummy piezoelectric element adjacent to the first piezoelectric element as being an electrical signal corresponding to an ultrasound echo signal received by the first piezoelectric element.
